(19) United States
(12) Patent Application Publication
(10) Pub. No.: US 2019/0204300 A1
(43) Pub. Date: Jul. 4, 2019

(54) IN VITRO METHOD FOR IDENTIFICATION AND ANALYSIS OF PROTEINS WITH STEM CELL FUNCTION USING A THREE-DIMENSIONAL CELL CULTURE MODEL OF THE SWEAT GLAND

(71) Applicant: Henkel AG & Co. KGaA, Duesseldorf (DE)

(72) Inventors: Patricia Klaka, Leverkusen (DE); Bernhard Banowski, Duesseldorf (DE); Sabine Gruedl, Erkelenz (DE); Thomas Welss, Duesseldorf (DE); Melanie Giesen, Geldern (DE)

(73) Assignee: Henkel AG & Co. KGaA, Duesseldorf (DE)

(21) Appl. No.: 16/331,421

(22) PCT Filed: Jul. 5, 2017
(86) PCT No.: PCT/EP2017/066778

§ 371 Date: Mar. 7, 2019

(30) Foreign Application Priority Data
Sep. 9, 2016 (DE) 10 2016 217 172.0

(51) Int. Cl. G01N 33/50; C12N 5/071

(52) U.S. Cl. CPC G01N 33/5082; C12N 5/0633; G01N 33/5073; C12N 5/0691; C12N 2501/10; C12N 2513/00; C12N 2533/54; C12N 2533/52; C12N 2533/74; G01N 2800/20

(57) ABSTRACT

The present disclosure concerns an in-vitro method for the identification and analysis of proteins with a stem cell function, in which initially, at least one three-dimensional sweat gland equivalent with from about 500 to about 500000 sweat gland cells as well as a diameter of from about 100 to about 6000 μm is provided and subsequently, proteins with a stem cell function in this equivalent are identified and analyzed. Preferably, in a further step c) of the method, the influence of test substances on the proteins previously identified in step b) is investigated. Because the three-dimensional sweat gland equivalents used in step a) comprise differently differentiated cells and emulate the in-vivo situation well, the measured data obtained with the in-vitro method as contemplated herein can readily be applied to the in-vivo situation.

### CROSS-REFERENCE TO RELATED APPLICATION

This application is a U.S. National-Stage entry under 35 U.S.C. § 371 based on International Application No. PCT/EP2017/066778, filed Jul. 5, 2017, which was published under PCT Article 21(2) and which claims priority to German Application No. 10 2016 217 172.0, filed Sep. 9, 2016, which are all hereby incorporated in their entirety by reference.

### TECHNICAL FIELD

The present disclosure relates to an in-vitro method for the identification and analysis of proteins with a stem cell function, in which initially, a three-dimensional sweat gland equivalent with from about 500 to about 500000 sweat gland cells as well as a diameter of from about 100 to about 6000 μm is provided and subsequently an identification and analysis of proteins with a stem cell function which are present in this equivalent is carried out. The three-dimensional sweat gland equivalents used as contemplated herein have ordered structures as well as differently differentiated cells and have a reactivity both as regards gene expression and also as regards protein expression to an external stimulus, for example a cholinergic stimulus by acetylcholine (also known as ACh).

### BACKGROUND

Washing, cleaning and care of an individual's body is a basic human necessity and modern industry is constantly looking out for ways to do justice to these human necessities in manifold manners. What is particularly important for daily hygiene is the sustained removal or at least reduction of body odor and armpit wetness. Armpit wetness and body odor arise because of secretion from eccrine and apocrine sweat glands in the human armpit. While the eccrine glands are responsible for the regulation of heat in the body and are responsible for the occurrence of armpit wetness, the apocrine glands exude a viscous secretion in reaction to stress, and an unpleasant body odor arises when it undergoes bacterial decomposition.

Initial research work on native eccrine and apocrine sweat glands were carried out as early as the beginning of the 20th century in order to classify them into the group of skin appendages belonging to the exocrine gland group. Thereafter, sweat glands were divided into apocrine and eccrine sweat glands as well as a hybrid of apocrine and eccrine sweat glands (also known as apoeccrine sweat glands). The forms mentioned above can be distinguished on the basis of their morphological and characteristic features.

The eccrine sweat gland, for example the human eccrine sweat gland, belongs to the unbranched coiled tubular glands and can be divided into the secretory base (also known as the coil), the dermal excretory duct (also known as the duct) and the epidermal duct (also known as the acrosyringium). The cells present in these sections of the gland have different purposes and functions such as, for example, secretion in the coil, reabsorption of ions in the duct as well as exuding the secretion, for example sweat, onto the surrounding skin through the acrosyringium. The eccrine sweat glands are primarily stimulated by the neurotransmitter acetylcholine (ACh), however a purinergic stimulation (for example with ATP/UTP) as well as an αβ-adrenergic stimulation (for example with noradrenaline) is also possible.

In respect of preventing armpit wetness and/or body odor, it is thus desirable to reduce and/or prevent secretions from eccrine and/or apocrine sweat glands. This may be carried out, for example, by obstructing the excretory ducts of eccrine sweat glands by what are known as plugs. In this regard, in the prior art, sweat-inhibiting aluminium and/or aluminium zirconium salts are used; however, these are no longer highly regarded by the consumer. Furthermore, antibacterial agents are used in the prior art which prevent the bacterial decomposition of sweat. However, such agents can have a negative influence on the natural microflora of the skin under the armpit. Thus, it would be apposite to provide cosmetic agents which are capable of reliably preventing armpit wetness and/or body odor and which are free from aluminium and/or aluminium-zirconium salts as well as acting as antibacterial agents. One possibility for preparing such agents arises from using substances which effectively inhibit the stimulation and/or the biological processes of the sweat glands and thus reduce or prevent the secretion of sweat. In order to be able to identify such substances, in-vivo tests with trial participants can be carried out. However, such tests are costly and are not suitable for high-throughput screening methods. On the other hand, in-vitro tests may be carried out using cell models of sweat glands on which the influence of test substances on stimulation of the sweat glands can be investigated.

So that the in-vitro test results can be properly applicable to the in-vivo situation, the cell model employed must emulate the in-vivo situation as closely as possible. For this, three-dimensional cell models are necessary, because the known two-dimensional models of the prior art are not physiologically close enough to native sweat glands, and are therefore poor imitators of the in-vivo situation. Moreover, an elucidation of the sweat secretion mechanism is required. This is because only in this manner can what are known as biological targets be identified, for example proteins produced by the sweat gland cells, which are influenced by the test substances to produce less sweat. A possible biological target which could be linked to sweat production are multipotent sweat gland cells which are already known in connection with processes in wound healing.

Thus, there is still a need for in-vitro methods with the aid of which biological targets which are responsible for an increased sweat production can be identified and analyzed. After the identification and analysis of such targets, an investigation of the influence of various test substances on these targets is carried out. In-vitro methods of this type should be capable of being standardized, should be inexpensive and should be rapid to carry out, so that the influence of the test substances on the biological targets can be determined using high throughput screening methods.

Thus, the aim of the present disclosure was to provide an in-vitro method for the identification and analysis of proteins with a stem cell function which is capable of being standardized, is inexpensive and rapid to carry out and the results therefrom should be applicable to the in-vivo situation.

### BRIEF SUMMARY

It has now surprisingly been discovered that, with the aid of specific three-dimensional sweat gland equivalents, an identification and analysis of proteins with a stem cell function is possible. The three-dimensional sweat gland equivalents used have an ordered structure. Furthermore, the primary sweat gland cells of these equivalents exhibit the same characteristics as native sweat glands. Thus, the measured data obtained with these equivalents in relation to the identification and analysis of proteins with a stem cell function are eminently applicable to the in-vivo situation.

Thus, in a first aspect, the present disclosure provides an in-vitro method for the identification and analysis of proteins with a stem cell function in the human sweat gland, the method comprising the following steps:
a) providing at least one three-dimensional sweat gland equivalent comprising from about 500 to about 500000 sweat gland cells, wherein the at least one three-dimensional sweat gland equivalent has a diameter of from about 100 to about 6000 μm, and
b) identification and analysis of at least one protein with a stem cell function in the at least one three-dimensional sweat gland equivalent provided in step a) of the method.

The three-dimensional sweat gland equivalents used in the method as contemplated herein form an ordered structure and have differentiated cells with the same characteristics as native sweat glands. Furthermore, these equivalents exhibit a reaction on the gene expression level as well as on the protein expression level to a stimulus by acetylcholine (ACh). The results obtained with the method as contemplated herein are therefore highly applicable to the in-vivo situation. By using cultured primary sweat gland cells during the production of the equivalents, good standardization can be obtained because a plurality of equivalents with the same properties can be produced from the cultured cells. Furthermore, by using cultured primary sweat gland cells, equivalents with almost identical numbers of sweat gland cells can be produced, which also ensures a high level of standardisability.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The term “protein with stem cell function” as contemplated herein should be understood to mean proteins which are formed from totipotent and pluripotent stem cells of the three-dimensional sweat gland equivalent and which, for example, play a role in wound healing processes. Such stem cells are non-specialized, continuously proliferating cells which contain all of the information of the whole organism and thus are capable of developing into more than one cell type under suitable conditions. They therefore can act in tissue-specific cell regeneration, for example during wound healing.

Furthermore, the term “three-dimensional sweat gland equivalent” as contemplated herein should be understood to mean a cell model formed from sweat gland cells which can extend in all three directions in space and in which the cells exhibit a similar function, for example an identical function to the cells of a native sweat gland.

In method step a) in the method as contemplated herein, at least one three-dimensional sweat gland equivalent with a specific cell count and a specific diameter is initially provided.

Particularly preferred three-dimensional sweat gland equivalents have a specific diameter. Thus, as contemplated herein, advantageously, the at least one three-dimensional sweat gland equivalent provided in step a) of the method has a diameter of from about 100 to about 4000 μm, of from about 100 to about 2000 μm, or for example of from about 200 to about 1500 μm. The diameter of the spherical sweat gland equivalents used as contemplated herein may, for example, be measured by microscopic measurement employing “CellSens” software.

In the context of the present disclosure, the sweat gland equivalents used in step a) of the method are free from matrix compounds and/or supports. The term “matrix compounds” should be understood here to mean compounds which are capable of forming spatial networks. However, this does not include the substances produced and excreted by the cells themselves which are capable of forming spatial networks. Furthermore, the term “supports” in the context of the present disclosure means self-supporting substances which can act as a base or scaffold for the sweat gland cells. In accordance with a preferred embodiment of the present disclosure, the at least one three-dimensional sweat gland equivalent is free from matrix compounds and/or supports, for example free from matrix compounds and supports.

The term “free from” as contemplated herein should be understood to mean that the three-dimensional sweat gland equivalents contain less than about 1% by weight, with respect to the total weight of the three-dimensional sweat gland equivalent, of matrix compounds and/or supports. Thus, in the context of the present disclosure, advantageously, the three-dimensional sweat gland equivalents used in step a) of the method contain from 0 to about 1% by weight, from 0 to about 0.5% by weight, from 0 to about 0.2% by weight, or for example 0% by weight of matrix compounds and supports, with respect to the total weight of the three-dimensional sweat gland equivalent.

In this regard, it is particularly advantageous for the three-dimensional sweat gland equivalents used in step a) of the method to be free from specific matrix compounds and supports. Thus, preferably, the three-dimensional sweat gland equivalent does not contain any matrix compounds and/or supports which are selected from the group formed by collagens, for example collagen type I and/or type III and/or type IV, scleroproteins, gelatins, chitosans, glucosamines, glucosaminoglucans (GAG), heparin sulphate proteoglucans, sulphated glycoproteins, growth factors, cross-linked polysaccharides, cross-linked polypeptides, as well as mixtures thereof.

Particularly preferably, the three-dimensional sweat gland equivalent provided in step a) of the method is an equivalent of the eccrine and/or apocrine human sweat gland. Exemplary embodiments of the present disclosure at least one three-dimensional sweat gland equivalent provided in step a) of the method is a three-dimensional sweat gland equivalent of the eccrine and/or apocrine human sweat gland. Such sweat gland equivalents are particularly suitable for the identification and analysis of proteins with a stem cell function as well as for the determination of the influence of test substances on these proteins.

Furthermore, as contemplated herein, particularly preferably, the three-dimensional sweat gland equivalent provided in step a) of the method has been produced from human eccrine and/or apocrine sweat glands. Thus, in the context of the present disclosure, advantageously, the at least one three-dimensional sweat gland equivalent provided in step a) of the method is a three-dimensional sweat gland equivalent obtained from eccrine and/or apocrine native human sweat gland cells.

Moreover, as contemplated herein, it has been shown to be advantageous when the three-dimensional sweat gland equivalents provided in step a) of the method has at least one specific type of cell. The use of equivalents of this type results in a particularly good identification and analysis of proteins with a stem cell function. Exemplary embodiments of the present disclosure at least one three-dimensional sweat gland equivalent provided in step a) of the method contains at least one cell type selected from the group of (i) coil cells, for example clear cells, dark cells, as well as myoepithelial cells, (ii) duct cells, as well as (iii) mixtures thereof. The term “clear cells” as contemplated herein should be understood to mean cells which have a clear or colourless cytoplasm when stained with stains, for example with haematoxylin and eosin. Such “clear cells” are secretory cells of the epithelium, wherein the plasma membrane is heavily folded at the apical and lateral surface. The cytoplasm of these “clear cells” contains large quantities of glycogen as well as many mitochondria. The cells are in contact with the lumen. The aqueous components of sweat, which contain electrolytes and inorganic substances, are excreted from this cell type. In contrast, the “dark cells” mentioned above are cells with a vacuole which stains positively on acid mucopolysaccharides, and thus the cytoplasm can be stained by stains. These “dark cells” are in contact with the basal membrane and have only a few mitochondria compared with “clear cells”. Macromolecules such as glycoproteins, for example, are excreted from these “dark cells”. The “myoepithelial cells” mentioned above should be understood to mean contractile epithelial cells which have a cytoskeleton with what are known as gap junctions and which can therefore contract. This promotes the exudation of secretions from the end parts of the glands. Cells of this type are found between the basal membrane and the aforementioned “clear cells” and “dark cells”. Finally, the term “duct cells” as contemplated herein should be understood to mean cells which form the wall of the duct and have a stratified cuboidal epithelium. The aforementioned cell types may be assayed by using immunocytochemical staining employing specific markers for these cells, in addition to using haematoxylin and eosin. A marker which is specific for myoepithelial cells is alpha-smooth muscle actin (also known as α-SMA). An example of a specific marker for “clear cells” is Substance P, and also S100. Furthermore, the marker known as CGRP (calcitonin-gene related peptide) may be used for “dark cells”, and for duct cells, the specific markers cytokeratin 10 (also known as CK10) and CD200 may be used.

Particularly preferred three-dimensional sweat gland equivalents used in step a) of the method will be described below.

Thus, in a particularly preferred embodiment of this aspect of the present disclosure, a three-dimensional sweat gland equivalent of the eccrine and/or apocrine human sweat gland is provided, comprising from about 500 to about 500000 sweat gland cells, wherein the three-dimensional sweat gland equivalent has a diameter of from about 200 to about 1500 μm.

Furthermore, in a particularly preferred embodiment of this aspect of the present disclosure, a three-dimensional sweat gland equivalent obtained from eccrine and/or apocrine native human sweat gland cells is provided, comprising from about 500 to about 500000 sweat gland cells, wherein the three-dimensional sweat gland equivalent has a diameter of from about 200 to about 1500 μm.

Moreover, in a particularly preferred embodiment of this aspect of the present disclosure, a three-dimensional sweat gland equivalent is provided comprising from about 500 to about 500000 sweat gland cells, wherein the three-dimensional sweat gland equivalent has a diameter of from about 200 to about 1500 μm and contains at least one cell type selected from the group formed by clear cells, dark cells, myoepithelial cells, duct cells as well as mixtures thereof.

In addition, in a particularly preferred embodiment of this aspect of the present disclosure, a three-dimensional sweat gland equivalent obtained from eccrine and/or apocrine native human sweat gland cells is provided, comprising from about 500 to about 500000 sweat gland cells, wherein the three-dimensional sweat gland equivalent has a diameter of from about 200 to about 1500 μm and contains at least one cell type selected from the group formed by clear cells, dark cells, myoepithelial cells, duct cells as well as mixtures thereof.

Furthermore, in a particularly preferred embodiment of this aspect of the present disclosure, a three-dimensional sweat gland equivalent of the eccrine and/or apocrine native human sweat gland cells is provided, comprising from about 500 to about 500000 sweat gland cells, wherein the three-dimensional sweat gland equivalent has a diameter of from about 200 to about 1500 μm and contains 0% by weight, with respect to the total weight of the three-dimensional sweat gland equivalent, of matrix compounds and supports.

In addition, in a particularly preferred embodiment of this aspect of the present disclosure, a three-dimensional sweat gland equivalent of the eccrine and/or apocrine native human sweat gland cells is provided, comprising from about 500 to about 500000 sweat gland cells, wherein the three-dimensional sweat gland equivalent has a diameter of from about 200 to about 1500 μm and contains 0% by weight, with respect to the total weight of the three-dimensional sweat gland equivalent, of matrix compounds and supports, wherein the matrix compounds and/or supports are selected from the group formed by collagens, for example collagen type I and/or type III and/or type IV, scleroproteins, gelatins, chitosans, glucosamines, glucosaminoglucans (GAG), heparin sulphate proteoglucans, sulphated glycoproteins, growth factors, crosslinked polysaccharides, crosslinked polypeptides and mixtures thereof.

The three-dimensional sweat gland equivalents provided in step a) of the method are more capable of standardization and more available than isolated sweat glands and resemble the in-vivo situation more closely than one-dimensional and two-dimensional sweat gland models. Furthermore, these equivalents constitute an inexpensive alternative to in-vivo studies in humans because, by employing these equivalents, proteins with a stem cell function can be identified and their influence on the secretion of sweat can be analyzed. This is because the three-dimensional sweat gland equivalents emulate in-vivo sweat glands both as regards their structure and also as regards their histological composition, so that the information obtained with these equivalents can readily be applied to humans.

The three-dimensional sweat gland equivalents provided in step a) of the method may, for example, be obtained using the following production method.

In a first step, isolated sweat glands are initially provided; they may be obtained from skin biopsies or the like and have been removed from their natural environment. Preferably, the sweat glands isolated in the first step are obtained by isolating native sweat glands, for example native eccrine and/or apocrine sweat glands, from human skin, wherein the isolation of the native sweat glands is carried out by employing enzymatic digestion of the human skin using a mixture of from about 2 to about 3 mg/mL of collagenase II and from about 0.1 to about 0.2 mg/mL of thermolysin for from about 3 to about 6 hours at from about 35° to about 40° C., for example at about 37° C.

In a second step, these isolated sweat glands are then cultured in a specific nutrient medium in order to obtain a cell culture. Particularly good culture of the isolated sweat gland cells obtained in the first step is obtained when a mixture formed by DMEM and Ham's F12 is used as the nutrient medium in a weight ratio of about 3:1, additionally containing about 10% by weight of foetal calf serum (FCS) with respect to the total weight of the mixture. Culturing of these cells in the nutrient medium described above is carried out for from about 7 to about 28 days, for example for about 14 days, at a temperature of from about 36° to about 38° C. and with a CO₂ content of about 5% by weight with respect to the total weight of the atmosphere employed for culturing.

In a third step, a cell preparation of primary sweat gland cells is produced from the cultured cells in a nutrient medium, wherein the cell count of the primary sweat gland cells in the cell preparation is from about 50 to about 25000 cells per μL, from about 100 to about 10000 cells per μL, from about 150 to about 5000 cells per μL, more from about 200 to about 3200 cells per μL, yet more from about 300 to about 1000 cells per μL, or for example from about 400 to about 600 cells per μL of nutrient medium. The cell preparation of primary sweat gland cells is produced by dissociation of the sweat gland cells cultured in the second step, for example by gentle trypsinization, culturing of these dissociated sweat gland cells in monolayer cultures, suspension of the cultured primary sweat gland cells in a nutrient medium, as well as adjusting the cell count. To culture the dissociated sweat gland cells and also to produce the cell suspension, it has been shown to be advantageous for the mixture of DMEM and Ham's F12 in a ratio by weight of about 3:1 which additionally contains about 10% by weight with respect to the total weight of the mixture of foetal calf serum (FCS) to be used as the nutrient medium. Culturing of the dissociated sweat gland cells is carried out at a temperature of from about 36° to about 38° C. and with a CO₂ content of about 5% by weight with respect to the total weight of the atmosphere employed for culture until confluence occurs.

Subsequently, in a fourth step, from about 10 to about 100 μL, from about 20 to about 80 μL, more from about 30 to about 70 μL, for example from about 40 to about 60 μL of this cell preparation is cultured using the hanging drop technique, i.e. in the form of a droplet suspended freely below a surface, until the three-dimensional sweat gland equivalents had been formed. In this connection, the use of what are known as hanging drop wells has been shown to be advantageous, as described, for example, in the published application WO 2012/014047 A1 and commercially available from Insphero as GravityPLUS® plates with the SureDrop® inlet dispensing system as well as GravityTRAP® plates for harvesting. For example, the cell preparation is cultured using the hanging drop technique for a time period of from about 1 to about 25 days, for example from about 2 to about 7 days, at a temperature of from about 36° to about 38° C. and with a CO₂ content of about 5% by weight with respect to the total weight of the atmosphere employed for culture. In this regard, during the culturing period, for example after from about 1 to about 3 days, about 40% by volume with respect to the total volume of the aforementioned cell preparation of the nutrient medium of the cell preparation is replaced with fresh nutrient medium.

After isolating the equivalents obtained by adding from about 50 to about 200 μL, for example from about 70 to about 100 μL of nutrient medium, the equivalents may be used directly for the method step b) in the method as contemplated herein, or be cultured anew. The new culture of the equivalents obtained is carried out for a time period of from about 1 to about 6 days at a temperature of from about 36° to about 38° C. and with a CO₂ content of about 5% by weight with respect to the total weight of the atmosphere employed for culture.

For example, the preparation of the three-dimensional sweat gland equivalents in step a) of the method is therefore carried out in the method described below which comprises the following steps in the order specified:
(i) providing isolated sweat glands, wherein the isolated sweat glands are obtained by isolation of native eccrine and/or apocrine sweat glands from human skin, and subsequent suspension of these isolated sweat glands in nutrient medium,
(ii) providing a cell preparation of primary sweat gland cells from the sweat glands isolated in step (i) of the method, wherein the cell count of the primary sweat gland cells in the cell preparation is from about 400 to about 600 cells per μL and wherein the cell preparation of primary sweat gland cells has a volume of from about 40 to about 60 μL,
(iii) culturing the cell preparation provided in step (ii) of the method using a hanging drop technique, wherein the suspended state of the cell preparation is achieved by using a hanging drop multi-well plate and wherein during the culturing period, about 40% by volume of the nutrient medium of the cell preparation with respect to the total volume of the cell preparation used in this step of the method is replaced by fresh nutrient medium,
(iv) isolating the three-dimensional sweat gland equivalent obtained in step (iii) of the method, wherein the isolation of the three-dimensional sweat gland equivalent is carried out by adding from about 50 to about 200 μL of nutrient medium in order to dissociate the model,
(v) optionally, culturing the three-dimensional sweat gland equivalent isolated in step (iv) of the method for a time period of from about 1 to about 6 days at a temperature of from about 36° to about 38° C. and with a CO₂ content of about 5% by weight with respect to the total weight of the atmosphere employed for culture.

Because in the context of the present disclosure, proteins with a stem cell function of the eccrine and/or apocrine human sweat gland are identified and analyzed, the equivalents provided in step a) are produced using eccrine and/or apocrine native human sweat glands. The term “eccrine and/or apocrine native sweat glands” as used herein should be understood to mean eccrine and/or apocrine sweat glands which have been isolated from human skin, for example from skin biopsies from humans or by other methods.

Furthermore, the three-dimensional sweat gland equivalents provided in step a) are produced exclusively with the use of in-vitro methods. As a consequence, the method contains no steps in which in-vivo methods are employed. Therefore, these equivalents may also be used to test substances which are provided for cosmetic use. Furthermore, this production method permits the inexpensive production of standardized equivalents to be carried out which can be used in high throughput screening methods. In addition, this production method results in three-dimensional sweat gland equivalents which form ordered structures, have differently differentiated cells and express sweat gland-specific markers so that good applicability of the in-vitro data to the in-vivo situation is made possible.

A method for the production of the three-dimensional sweat gland equivalents provided in step a) of the method as contemplated herein is disclosed, for example, in the German application DE 10 2015 222 279, the content of which is incorporated herein by reference.

In the second step of the method as contemplated herein, the identification and analysis of at least one protein with a stem cell function in the three-dimensional sweat gland equivalent provided in step a) of the method is carried out.

In the context of the present disclosure, advantageously, specific proteins with a stem cell function are identified and analyzed. Exemplary embodiments of the present disclosure in step b) of the method, the at least one protein with a stem cell function is selected from the group formed by structural proteins, signalling proteins, cell proliferation proteins, cell adhesion proteins as well as mixtures thereof. The term “structural proteins” as used as contemplated herein describes proteins which act as a scaffold material in cells and are vital to the construction of fibres by the aggregation of monomeric protein strands. They are thus also described as scleroproteins, fibrous proteins or scaffold proteins and act to stabilize the shape, strength and elasticity of cells. The term “signalling proteins” as used as contemplated herein should be understood to mean proteins which transfer signals to the target cell by interaction with the receptor of a target cell or by penetration into the cell through the cell membrane. After activation of the target cell, the activation of what are known as “second messengers” usually occurs, leading to various physiological effects. Signalling proteins may, for example, be selected from proteins with lipid residues, phospholipids, amino acids, monoamines, proteins, glycoproteins or gases. While signalling proteins which bind to receptors on the cell surface usually have a high molecular weight and are hydrophilic, the signalling proteins which penetrate into the cell usually have low molecular weights and are hydrophobic. As contemplated herein, cell proliferation proteins are proteins which control cell proliferation, for example increase it or reduce it. The term “cell proliferation” as used here should be understood to mean increasing the cell count due to cell growth and cell division. In contrast, cell adhesion proteins in the context of the present disclosure are proteins which are located on the cell surface and are responsible for binding to other cells or to the extracellular matrix. By employing these proteins, then, the cells bind together, and also the cells can bind to their environment. Proteins of this type have three domains, the intracellular domain, the transmembrane domain as well as the extracellular domain. While the intracellular domain interacts with the cytoskeleton, the extracellular domain binds either to other cell adhesion proteins of the same type or to cell adhesion proteins of the extracellular matrix.

The aforementioned proteins not only have an influence on wound healing, but can also influence sweat secretion. Thus, these proteins are particularly suitable as biological targets for the investigation of the secretion mechanism.

As contemplated herein, the identification and analysis of proteins with a stem cell function, for example the aforementioned proteins, is carried out using specific methods. Thus, as contemplated herein, preferably, the identification and analysis in step b) of the method is carried out using methods selected from the group formed by molecular biological methods, protein analyzes, assays to determine the functionality, as well as combinations thereof. In the context of the present disclosure, examples of molecular biological methods which may be used are NGS (next generation sequencing) analysis, as well as qRT-PCR (quantitative real-time PCR). The aforementioned proteins may be identified and quantitatively assayed by employing gene expression analyzes. The protein expression level obtained in the three-dimensional sweat gland equivalents was compared with the expression level of these proteins in samples of human sweat glands as well as in full skin samples. The expression level of these proteins in the three-dimensional sweat gland equivalents as well as in the human sweat gland was significantly higher than in the samples of full skin, so that these proteins therefore can constitute specific marker proteins for the sweat gland. Furthermore, the expression of these proteins obtained in the three-dimensional sweat gland equivalents were comparable with the expression of these proteins in the human sweat glands. The sweat gland equivalents used in the method as contemplated herein therefore simulate the in-vivo situation superbly and in this manner, ensure good applicability of the in-vitro results to the in-vivo situation.

Examples of suitable protein analyzes are immunomarking of the aforementioned proteins using specific markers such as immunofluorescence methods, Western Blot analyzes and/or ELISA. The two latterly cited methods may in fact also be used to carry out a quantitative determination of the aforementioned proteins.

In the context of the method as contemplated herein, it has been shown to be advantageous for a further step c) of the method to be carried out after the step b) of the method. In this step c) of the method, the influence of various test substances on the proteins with a stem cell function identified in step b) of the method is determined, for example that of the previous determined proteins. Exemplary embodiments of the present disclosure in an additional step c) of the method, the influence of compounds on the proteins with a stem cell activity identified in step b) of the method is investigated. Preferably, the compounds used in step c) of the method are inhibitors of these proteins in the case in which the proteins increase the secretion of sweat. However, if the aforementioned proteins reduce the secretion of sweat, then preferably, activators are used in the compounds in step c) of the method. In this step of the method, in addition to the influence of the compounds on the secretion of sweat, the influence of these compounds on the stem cell activity and on wound healing may also be investigated.

In this connection, preferably, the methods specified in step c) of the method are used to determine the influence of the compound on the proteins identified in step b) of the method. Thus, as contemplated herein, advantageously, in step c) of the method, the influence of the at least one compound is investigated using methods which are selected from the group formed by molecular biological methods, protein analyses, assays to determine the functionality as well as combinations thereof. Regarding the methods, reference should be made to the aforementioned methods which are used in step b) of the method which may equally be employed for carrying out step c) of the method.

The following examples illustrate the present disclosure without, however, limiting its scope:

### Examples

### 1 Production of the Three-Dimensional Sweat Gland Equivalents (Step a) of the Method)

### 1.1 Isolation of Sweat Glands

The native sweat glands were obtained from human tissue samples, what are known as biopsies, taken from patients undergoing plastic surgery and who had agreed that the material could be used for research purposes. The tissue used was removed during upper arm lifts and facelifts. The eccrine and apocrine sweat glands from the armpit region were isolated from these.

To this end, the respective biopsy was divided into small pieces and thereafter cut into pieces with a maximum size of approximately 1 cm×1 cm. Next, the skin was digested with a mixture of equal parts of collagenase II (5 mg/mL) and thermolysin (0.25 mg/mL) at 37° C. in an incubator for approximately 3.5 to 5 hours until the connective tissue had been almost completely digested. This mixture was then centrifuged at 1200 rpm for 5 minutes and the supernatant was discarded in order to remove the enzyme solution as well as any surplus fat. The pellet which remained was taken up in DMEM solution and the solution was transferred into a petri dish. Intact sweat glands were isolated under a binocular microscope using a microcapillary and transferred into fresh DMEM medium.

### 1.2 Culturing of Isolated Native Sweat Glands

The sweat glands isolated in step 1.1 were placed in culture flasks coated with collagen I and then 25 mL of nutrient medium was added. After culturing for 7 to 21 days in an incubator at 37° C. and under 5% CO₂, the grown sweat gland cells were dissociated and cultured again to confluence (monolayer culture of primary sweat gland cells) in culture flasks coated with collagen I.

The composition of the nutrient medium used was as follows:

| Components of medium | | |
|---|---|---|
| DMEM/Ham's F12 Nutrient Mix | 3:1 | |
| Foetal Calf Serum (FCS) | 10% | |
| EGF | 10 | ng/mL |
| Hydrocortisone | 0.4 | μg/mL |
| Insulin | 0.12 | UI/mL |
| Choleratoxin | 10⁻¹⁰ | M |
| Adenine | 2.43 | g/mL |
| Gentamicin | 25 | μg/mL |
| Penicillin G | 100 | UI/mL |
| Triiodothyronine | 2 * 10⁻⁹ | M |
| Ascorbyl-2-phosphate | 1 | mM |

### 1.3 Production of the Cell Preparation and of the Three-Dimensional Sweat Gland Equivalents

After determining the exact cell counts of the above monolayer cultures of the primary sweat gland cells, they were adjusted to a cell count of 10 to 5000 cells per μL using the above nutrient medium, and then 50 μL of this cell suspension was transferred using the “SureDrop® Inlet” system into each well of a “GravityPLUS®” plate (both from Insphero AG, Switzerland). Culturing was carried out at 36° to 38° C. and under a CO₂ content of 5% by weight with respect to the total weight of the atmosphere used for culture. After 1 to 3 days, 40% by weight of the medium in each of the wells of the “GravityPLUS®” plate was replaced with fresh nutrient medium. After 3 to 5 days of culture, the 3D sweat gland equivalents were harvested by adding 50 to 200 μL of nutrient medium and transferred into a “GravityTRAP®” plate (Insphero AG, Switzerland). Prior to harvesting, the “GravityTRAP®” plate was moistened with 60 to 100 μL of keratinocyte medium with the aid of a multi-channel pipette in order to minimize the formation of air bubbles and to prevent loss of the three-dimensional sweat gland equivalents. After harvesting, the plate was centrifuged for 1 to 5 minutes at 100 to 300×g in order to remove air bubbles. A portion of the three-dimensional sweat gland equivalents was analyzed, but a further portion was cultured for a further 1 to 6 days in the wells of the harvesting plate at 37° C. and with 5% by weight of CO₂ with respect to the total weight of the atmosphere used for culture.

2. Identification and Analysis of a Protein with a Stem Cell Function (Step b) of the Method)

The detection of the aforementioned structural proteins, signalling proteins, cell proliferation proteins, cell adhesion proteins may be carried out, for example, by employing molecular biological methods. To this end, firstly, the mRNA was isolated with the aid of the “RNeasy Micro Kit” (Qiagen) in accordance with the manufacturer's instructions and subsequently analyzed using quantitative Real Time PCR (Bellas et. al.: “In Vitro 3D Full-Thickness Skin-Equivalent Tissue Model Using Silk and Collagen Biomaterials”; Macromolecular Bioscience, 2012, 12, pages 1627-1636). However, it is also possible to assay the aforementioned proteins with a stem cell function with the aid of immunofluorescence staining. Thus, for example, the structural protein (also known as the intermediate filament) nestin could be assayed in the three-dimensional sweat gland equivalents provided in step a) of the method. Nestin is expressed in 90% of all stem cells derived from sweat glands (also known as SCSCs) and this is therefore an indicator of the presence of stem cells in the three-dimensional sweat gland equivalents.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An in-vitro method for the identification and analysis of proteins with a stem cell function in the human sweat gland, the method comprising the following steps:
a) providing at least one three-dimensional sweat gland equivalent comprising from about 500 to about 500000 sweat gland cells, wherein the at least one three-dimensional sweat gland equivalent has a diameter of from about 100 to about 6000 μm, and
b) identifying and analysing at least one protein with a stem cell function in the at least one three-dimensional sweat gland equivalent provided in step a) of the method.

2. The method as claimed in claim 1, wherein the at least one three-dimensional sweat gland equivalent provided in step a) of the method has a diameter of from about 100 to about 4000 μm.

3. The method as claimed in claim 1, wherein the at least one three-dimensional sweat gland equivalent provided in step a) of the method is free from matrix compounds and/or supports.

4. The method as claimed in claim 3, wherein the matrix compounds and/or supports are selected from the group formed by collagens, scleroproteins, gelatins, chitosans, glucosamines, glucosaminoglucans (GAG), heparin sulphate proteoglucans, sulphated glycoproteins, growth factors, crosslinked polysaccharides, crosslinked polypeptides and mixtures thereof.

5. The method as claimed in claim 1, wherein the at least one three-dimensional sweat gland equivalent provided in step a) of the method is a three-dimensional sweat gland equivalent of the eccrine and/or apocrine human sweat gland.

6. The method as claimed in claim 1, wherein the at least one three-dimensional sweat gland equivalent provided in step a) of the method comprises at least one cell type selected from the group formed by (i) coil cells, (ii) duct cells, as well as (iii) mixtures thereof.

7. The method as claimed in claim 1, wherein in step b) of the method, the at least one protein with a stem cell function is selected from the group formed by structural proteins, signalling proteins, cell proliferation proteins, cell adhesion proteins as well as mixtures thereof.

8. The method as claimed in claim 1, wherein the identification and analysis in step b) of the method is carried out using methods selected from the group formed by molecular biological methods, protein analyses, assays to determine the functionality, as well as combinations thereof.

9. The method as claimed in claim 1, wherein in an additional step c) of the method, the influence of compounds on the proteins with a stem cell activity identified in step b) of the method is investigated.

10. The method as claimed in claim 9, wherein in step c) of the method, the influence of the at least one compound is investigated using methods which are selected from the group formed by molecular biological methods, protein analyses, assays to determine the functionality, as well as combinations thereof.

11. The method as claimed in claim 1, wherein the at least one three-dimensional sweat gland equivalent provided in step a) of the method has a diameter of from about 100 to about 2000 μm.

12. The method as claimed in claim 1, wherein the at least one three-dimensional sweat gland equivalent provided in step a) of the method has a diameter of from about 200 to about 1500 μm.

13. The method as claimed in claim 1, wherein the at least one three-dimensional sweat gland equivalent provided in step a) of the method is free from matrix compounds and supports.

14. The method as claimed in claim 13, wherein the matrix compounds and supports are selected from the group formed by collagen type I and/or type III and/or type IV, scleroproteins, gelatins, chitosans, glucosamines, glucosaminoglucans (GAG), heparin sulphate proteoglucans, sulphated glycoproteins, growth factors, crosslinked polysaccharides, crosslinked polypeptides and mixtures thereof.

15. The method as claimed in claim 1, wherein the at least one three-dimensional sweat gland equivalent provided in step a) of the method comprises at least one cell type selected from the group formed by (i) clear cells, dark cells, as well as myoepithelial cells, (ii) duct cells, as well as (iii) mixtures thereof.
